(19) United States
(12) Patent Application Publication
(10) Pub. No.: US 2025/0136946 A1
(43) Pub. Date: May 1, 2025

(54) REAL-TIME CHEMICAL SCREENING METHOD USING LIVER ORGANOIDS FLUORESCENTLY LABELED WITH CYTOCHROME P450 1A1 ENZYME

(71) Applicant: Korea Research Institute of Chemical Technology, Daejeon (KR)

(72) Inventors: Hyemin KIM, Daejeon (KR); Han-Jin PARK, Daejeon (KR); Ji-Woo KIM, Daejeon (KR); Haneul NOH, Daejeon (KR)

(73) Assignee: Korea Research Institute of Chemical Technology, Daejeon (KR)

(21) Appl. No.: 18/927,121

(22) Filed: Oct. 25, 2024

(30) Foreign Application Priority Data
Oct. 25, 2023 (KR) 10-2023-0143934
Sep. 23, 2024 (KR) 10-2024-0128433

(51) Int. Cl. C12N 5/071; G01N 33/58

(52) U.S. Cl. CPC C12N 5/0671; G01N 33/582; C12N 2503/04; C12N 2506/45

(57) ABSTRACT

The present disclosure relates to a real-time chemical screening method using hepatic organoids fluorescently labeled with cytochrome P450 1A1 enzyme. The method for screening an AHR modulator or a hazardous chemical using CYP1A1 fluorescently labelled human pluripotent stem cell line-derived hepatic organoids according to the present disclosure enables the detection of substances that induce CYP1A1 regulation with higher sensitivity than when using the conventional human pluripotent stem cell line-derived hepatocytes by using hepatic organoids that can mimic the human body, and thus can be usefully utilized for early screening of new toxic or carcinogenic compounds while replacing animal testing.

### CROSS-REFERENCE TO RELATED APPLICATION(S)

This application claims benefit of priority to Korean Patent Application No. 10-2023-0143934 filed on Oct. 25, 2023, and Korean Patent Application No. 10-2024-0128433 filed on Sep. 23, 2024, in the Korean Intellectual Property Office, the disclosure of which is incorporated herein by reference in its entirety.

### REFERENCE TO AN ELECTRONIC SEQUENCE LISTING

The contents of the Sequence Listing XML (sequence listing.xml; Date of Creation: Oct. 25, 2024; and Size: 3,634 bytes) is herein incorporated by reference in its entirety.

### BACKGROUND

### 1. Field

The present disclosure relates to a real-time chemical screening method using hepatic organoids fluorescently labeled with cytochrome P450 1A1 enzyme.

### 2. Description of Related Art

Benzo[a]pyrene (BaP), a representative class 1 carcinogen in daily life, is produced a variety of reasons, including coal tar, cigarette smoke, automobile exhaust, etc., the composition of carbohydrates, proteins, and fats, which are main components of food, during cooking and processing of food, and the burning of all organic substances, and causes various diseases such as colon cancer, breast cancer, skin cancer, and endocrine disorders due to genetic mutation and carcinogenicity. In addition, dioxin (2,3,7,8-tetrachlorodibenzo-p-dioxin, TCDD), a main component of defoliants used during the Vietnam War, is also produced by burning oil or tobacco, or in factories that make chemicals such as pesticides, and is most commonly produced when plastic or vinyl is burned, which has various negative effects on the human body, including lung cancer, liver cancer, reproductive disorders, immune system damage, and hormonal imbalances.

On the other hand, an aryl hydrocarbon receptor (AHR) is a ligand-activated transcription factor that functions as a sensor of extracellular signals and environmental ligands and mediates numerous toxicological consequences associated with various environmental xenobiotics, and binds with high affinity to various carcinogens such as TCDD and BaP, allowing the carcinogens to begin their action in the human body.

The affinity of AHR with carcinogens is related to the degree of AHR activation and toxicity induction, and is used as an indicator of AHR activity against toxicity and carcinogens in the case of cytochrome P450, particularly CYP1A1, which detoxifies the liver by converting drugs or toxic substances into water-soluble substances in the liver and excreting them. These AHR signaling pathways are known to be involved in the development and differentiation of immune cells, including T and B cells as well as antigen-presenting cells such as dendritic cells and macrophages. In addition, evidence has been accumulated on the role of the AHR in cancer progression and the therapeutic potential of selective AHR modulators with agonistic or antagonistic activity, confirming the potential of the AHR as a novel drug target for cancer therapy.

Currently, there are more than 50,000 chemicals in the world, but only less than 10% of them have been confirmed to be harmful through toxicity assessment using animal testing, etc. Therefore, there is an increasing need for the discovery of carcinogenic or toxic substances and methods for screening AHR modulators. However, there are structural differences in the AHR between experimental animals and humans used to discover these carcinogens or toxic substances and screen for AHR regulators, resulting in a difference in affinity between the AHR and carcinogens, a difference in CYP1A1 activity, and eventually a difference in the degree of toxicity induction, so animal testing alone has limitations in accurately predicting and evaluating the toxicity or carcinogenicity of a specific substances in humans. Therefore, there is a need to develop technologies that may replace animal testing and accurately screen for new toxic or carcinogenic substances, hazardous chemicals, or AHR modulators, etc. that mimic the human body.

### Related Art Document

### Patent Document

(Patent Document 0001) Korean Patent Registration No. 10-2400211

### SUMMARY

An object of the present disclosure is to solve the aforementioned problems and other problems related thereto.

An object of the present disclosure is to provide a method for screening an aryl hydrocarbon receptor (AHR) modulator or a hazardous chemical, comprising: treating hepatic organoids derived from a human pluripotent stem cell line expressing cytochrome P450 fused to a fluorescent protein, with a test substance; and measuring a signal intensity of the fluorescent protein.

Another object of the present disclosure is to provide a composition for screening an AHR modulator or a hazardous chemical, comprising hepatic organoids derived from a human pluripotent stem cell line expressing cytochrome P450 fused to a fluorescent protein.

The technical problem to be accomplished in accordance with the technical idea of the present disclosure disclosed in the specification are not limited to the problem for solving the above-mention problems, and other problems that are not mentioned may be obviously understood by those skilled in the art from the following description.

This will be described in detail as follows. Meanwhile, each of the descriptions and embodiments disclosed in the present application may also be applied to each other descriptions and embodiments. In other words, any combination of the various elements disclosed in the present application falls within the scope of the present application. In addition, the scope of the present application cannot be considered as being limited by the specific description set forth below.

In an aspect for achieving the object, the present disclosure provides a method for screening an aryl hydrocarbon receptor (AHR) modulator or a hazardous chemical, comprising: treating hepatic organoids derived from human pluripotent stem cell lines expressing cytochrome P450 fused to a fluorescent protein, with a test substance; and measuring a signal intensity of the fluorescent protein.

In the present disclosure, the “human pluripotent stem cells” refer to stem cells capable of differentiating into almost any type of cells that constitutes the endoderm, mesoderm, or ectoderm.

In the present disclosure, the “human pluripotent stem cells” may be, but is not limited to, human embryonic stem cells or human induced pluripotent stem cells, and preferably human induced pluripotent stem cells.

In the present disclosure, the “induced pluripotent stem cells (iPSCs)” also known as reverse differentiation stem cells, refer to stem cells with pluripotency that are produced by introducing four specific genes (Oct3/4, Sox2, c-Myc, Klf4) that induce reverse differentiation into somatic cells, such as adult skin cells without pluripotency and then expressing them, or by extracting a reverse differentiation-induced proteins created from the cells into which the four genes that induce reverse differentiation have been introduced and then infusing them back into the somatic cells. The induced pluripotent stem cells have the advantage of solving the ethical problems of conventional embryonic stem cell research, where stem cells can only be obtained by destroying embryos developing from fertilized eggs, and of having no problem with immune rejection.

In the present disclosure, the “hepatic organoids” may express cytochrome P450 labeled with a fluorescent protein.

In the present disclosure, the hepatic organoids derived from the human pluripotent stem cells may be hepatic organoids matured by culture in a medium containing no extracellular matrix.

In the present disclosure, the “extracellular matrix” refers to an assembly of biopolymers that fill the gaps between cells to physically support tissues or surround cells to create an environment for cells to live robustly. The extracellular matrix of the present disclosure may be, but is not limited to, Matrigel, Cultrex Basement Membrane Extract (BME), or Geltrex Matrix, preferably Matrigel, and more specifically Matrigel GFR (Growth Factor Reduced).

The hepatic organoids may be matured into hepatic organoids (HOs) by culturing in a medium containing no extracellular matrix, hepatic endoderm organoids (HEOs) obtained by the following steps comprising: (i) differentiating human pluripotent stem cells (hPSCs) into definitive endoderm (DE) cells; (ii) differentiating the definitive endoderm cells in the step (i) into hepatic endoderm (HE) cells; and (iii) differentiating the hepatic endoderm cells in the step (ii) into hepatic endoderm organoids (HEOs).

In the present disclosure, the method may further include: subculturing the hepatic endoderm organoids, prior to culturing the hepatic organoids in a medium containing no extracellular matrix. In addition, the hepatic endoderm organoids cultured in the medium containing no extracellular matrix may be, but are not limited to, single cells.

In the step of subculturing the hepatic endoderm organoids and then maturing the hepatic endoderm organoids into hepatic organoids, the hepatic endoderm organoids may be added to an expansion medium (EM) containing no extracellular matrix, and cultured in a bioreactor at 37° C. and 5% CO₂ for 4 to 8 days, preferably for 6 days. In addition, the culture may be carried out in the bioreactor at 40 to 100 rpm, preferably at 50 to 90 rpm, and more preferably at 80 rpm.

The EM containing no extracellular matrix may specifically contain, but is not limited to, fibroblast growth factor 10 (FGF10), hepatocyte growth factor (HGF), nicotinamide, [Leu¹⁵]-Gastrin I human, N-acetyl-L-cysteine, A83-01, Forskolin, and CHIR99021.

The FGF10 may be contained in the EM at, but is not limited to, 30 to 70 ng/ml, preferably 40 to 60 ng/ml, and more preferably 50 ng/ml.

The HGF may be contained in the EM at, but is not limited to, 5 to 45 ng/ml, preferably 15 to 35 ng/ml, and more preferably 25 ng/ml.

The nicotinamide may be contained in the EM at, but is not limited to, 1 to 20 mM, preferably 5 to 15 mM, more preferably 10 mM.

The [Leu¹⁵]-Gastrin I human may be contained in the EM at, but is not limited to, 1 to 20 nM, preferably 5 to 15 nM, and more preferably 10 nM.

The N-acetyl-L-cysteine may be contained in the EM at, but is not limited to, 0.25 to 2.25 mM, preferably 0.5 to 2 mM, and more preferably 1.25 mM.

The A83-01 may be contained in the EM at, but is not limited to, 1 to 9 μM, preferably 3 to 7 μM, and more preferably 5 μM.

The Forskolin may be contained in the EM at, but is not limited to, 1 to 20 μM, preferably 5 to 15 μM, and more preferably 10 μM.

The CHIR99021 may be contained in the EM at, but is not limited to, 1 to 5 μM, preferably 2 to 4 μM, and more preferably 3 μM.

After the culture, the method may include replacing the medium with a hepatic organoids differentiation medium (DM) and then performing an additional culture. Preferably, the DM may not contain extracellular matrix, and specifically, may not contain Matrigel GFR. The culture may be carried out at 37° C. and 5% CO₂ for 5 to 9 days, preferably for 6 to 8 days, and more preferably for 7 days, and the medium may be replaced every 2 to 4 days. In addition, the culture may be carried out in the bioreactor at 40 to 80 rpm, preferably at 50 to 70 rpm, and more preferably at 60 rpm.

The DM may contain, but is not limited to, bone morphogenetic protein 7 (BMP7), fibroblast growth factor 19 (FGF19), hepatocyte growth factor (HGF), N-acetyl-L-cysteine, A83-01, DAPT, and dexamethasone.

The BMP7 may be contained in the DM at, but is not limited to, 10 to 40 ng/ml, preferably 20 to 30 ng/ml, and more preferably 25 ng/ml.

The FGF19 may be contained in the DM at, but is not limited to, 80 to 120 ng/ml, preferably 90 to 110 ng/ml, and more preferably 100 ng/ml.

The HGF may be contained in the DM at, but is not limited to, 5 to 45 ng/ml, preferably 15 to 35 ng/ml, and more preferably 25 ng/ml.

The N-acetyl-L-cysteine may be contained in the DM at, but is not limited to, 0.25 to 2.25 mM, preferably 0.5 to 2 mM, and more preferably 1.25 mM.

The A83-01 may be contained in the DM at, but is not limited to, 0.1 to 0.9 μM, preferably 0.3 to 0.7 μM, and more preferably 0.5 μM.

The DAPT may be contained in the DM at, but is not limited to, 0.5 to 2 μl, and preferably 1 μl.

The dexamethasone may be contained in the DM at, but is not limited to, 1 to 5 μM, preferably 2 to 4 μM, and more preferably 3 μM.

The “endoderm” in the step (i) is one of the three germ layers of the early embryo stage, and forms the lining of the three appendage organs (teeth, tongue, and glandular organs) that develop towards the tail of the stomach.

The step (i) of differentiating into definitive endoderm cells may include, but is not limited to, culturing human pluripotent stem cells in mTeSR™-E8™ medium for 1 day.

The step (i) of differentiating into definitive endoderm cells may include: culturing human pluripotent stem cells cultured for 1 day in definitive endoderm (DE) differentiation medium 1 for 22 to 30 hours, and then culturing them in the definitive endoderm (DE) differentiation medium 2 for 3 to 6 days.

The endoderm (DE) differentiation medium 1 may contain, but is not limited to, RPMI-1640 containing 0.1% BSA, 1% B27 supplement, activin A, sodium butyrate, and CHIR99021.

The activin A may be contained in the endoderm differentiation medium 1 at, but is not limited to, 30 to 70 ng/ml, preferably 40 to 60 ng/ml, and more preferably 50 ng/ml.

The sodium butyrate may be contained in the endoderm differentiation medium 1 at, but is not limited to, 0.1 to 0.9 mM, preferably 0.3 to 0.7 mM, and more preferably 0.5 mM.

The CHIR99021 may be contained in the endoderm differentiation medium 1 at, but is not limited to, 1 to 5 uM, preferably 2 to 4 uM, and more preferably 3 uM.

The endoderm (DE) differentiation medium 2 may contain, but is not limited to, RPMI-1640 containing 0.1% BSA, 1% B27 supplement, activin A, and sodium butyrate.

The activin A may be contained in the endoderm differentiation medium 2 at, but is not limited to, 30 to 70 ng/ml, preferably 40 to 60 ng/ml, and more preferably 50 ng/ml.

The sodium butyrate may be contained in the endoderm differentiation medium 2 at, but is not limited to, 0.05 to 1.05 mM, preferably 0.09 to 0.11 mM, and more preferably 0.1 mM.

The step (ii) of differentiating into hepatic endoderm cells may include: culturing the endoderm cells in hepatic endoderm (HE) differentiation medium for 2 to 6 days, preferably 4 days, and may include medium replacement every 22 to 26 hours.

The hepatic endoderm (HE) differentiation medium may contain, but is not limited to, RPMI-1640 containing 0.1% BSA, 1% B27 supplement, Bone Morphogenetic Protein 4 (BMP4), and SB431542.

The BMP4 may be contained in the hepatic endoderm differentiation medium at, but is not limited to, 30 to 70 ng/ml, preferably 40 to 60 ng/ml, and more preferably 50 ng/ml.

The SB431542 may be contained in the hepatic endoderm differentiation medium at, but is not limited to, 5 to 15 μM, preferably 8 to 12 μM, and more preferably 10 μM.

After step (ii) of the differentiation, the epithelial cell adhesion molecule (EpCAM) positive population may be analyzed by flow cytometry, and if the EpCAM-positive cells are 95% or more, step (iii) may be proceeded to.

The step (iii) of differentiating into hepatic endoderm organoids (HEOs) may include: culturing the hepatic endoderm cells that have completed the differentiation in the step (ii) in the hepatic endoderm organoids generation medium (GM).

The GM may contain, but is not limited to, Advanced DMEM/F12 containing 10 mM hydroxyethyl piperazine ethane sulfonic acid (HEPES), 1% GlutaMAX, 100 U/ml penicillin-streptomycin, 0.1% BSA, 1% B27 supplement minus Vitamin A, 1% N2 supplement, fibroblast growth factor 10 (FGF10), hepatocyte growth factor (HGF), nicotinamide, [Leu¹⁵]-Gastrin I human, N-acetyl-L-cysteine, A83-01, Forskolin, CHIR99021, and Matrigel growth factor reduced (GFR).

The FGF10 may be contained in the GM at, but is not limited to, 30 to 70 ng/ml, preferably 40 to 60 ng/ml, and more preferably 50 ng/ml.

The HGF may be contained in the GM at, but is not limited to, 5 to 45 ng/ml, preferably 15 to 35 ng/ml, more preferably 25 ng/ml.

The nicotinamide may be contained in the GM at, but is not limited to, 1 to 20 mM, preferably 5 to 15 mM, and more preferably 10 mM.

The [Leu¹⁵]-Gastrin I human may be may be contained in the GM at, but is not limited to, 1 to 20 nM, preferably 5 to 15 nM, and more preferably 10 nM.

The N-acetyl-L-cysteine may be contained in the GM at, but is not limited to, 0.25 to 2.25 mM, preferably 0.5 to 2 mM, and more preferably 1.25 mM.

The A83-01 may be contained in the GM at, but is not limited to, 1 to 9 μM, preferably 3 to 7 μM, and more preferably 5 μM.

The Forskolin may be contained in the GM at, but is not limited to, 1 to 20 μM, preferably 5 to 15 μM, and more preferably 10 μM.

The CHIR99021 may be contained in the GM at, but is not limited to, 1 to 5 μM, preferably 2 to 4 μM, and more preferably 3 μM.

The Matrigel GFR may be contained in the GM at, but is not limited to, 0.2 to 0.8 mg/ml, preferably 0.3 to 0.7 mg/ml, and more preferably 0.5 mg/ml.

The culture of the hepatic endoderm cells in the GM may be carried out for 10 to 18 days, preferably for 12 to 16 days, and more preferably for 14 days. During the culture, the medium may be replaced every 2 to 4 days, preferably every 3 days.

After the step (iii), the method may further include: subculturing hepatic endoderm organoids.

The step of subculturing may include a process of spinning down at 800 to 1,200 rpm for 30 seconds to 2 minutes to remove the supernatant. The step of subculturing may further include: removing the supernatant, adding Dulbecco's phosphate-buffered saline (DPBS) to loosen the pellet, spinning down at 800 to 1,200 rpm for 30 seconds to 2 minutes to remove the supernatant once more, and adding 0.5 to 2 ml of enzyme to loosen the pellet. The enzyme may be TrypLE Express, Accutase, or 0.25% trypsin-EDTA, preferably TrypLE Express, and more preferably 1 ml of TrypLE Express. The step of subculturing may include a process of adding the enzyme to loosen the pellet, leaving it in an incubator at 37° C. and C02 for 8 to 12 minutes, pipetting again to loosen all the pellets, and spinning down to remove the supernatant.

The subculturing may include: removing the supernatant and then culturing the hepatic endoderm organoids in an expansion medium (EM) for hepatic endoderm organoids.

The EM may be the same as the GM. Specifically, the EM may contain, but is not limited to, fibroblast growth factor 10 (FGF10), hepatocyte growth factor (HGF), nicotinamide, [Leu¹⁵]-Gastrin I human, N-acetyl-L-cysteine, A83-01, Forskolin, CHIR99021, and Matrigel growth factor reduced (GFR).

The FGF10 may be contained in the EM at, but is not limited to, 30 to 70 ng/ml, preferably 40 to 60 ng/ml, and more preferably 50 ng/ml.

The HGF may be contained in the EM at, but is not limited to, 5 to 45 ng/ml, preferably 15 to 35 ng/ml, and more preferably 25 ng/ml.

The nicotinamide may be contained in the EM at, but is not limited to, 1 to 20 mM, preferably 5 to 15 mM, and more preferably 10 mM.

The [Leu¹⁵]-Gastrin I human may be contained in the EM at, but is not limited to, 1 to 20 nM, preferably 5 to 15 nM, and more preferably 10 nM.

The N-acetyl-L-cysteine may be contained in the EM at, but is not limited to,0.25 to 2.25 mM, preferably 0.5 to 2 mM, and more preferably 1.25 mM.

The A83-01 may be contained in the EM at, but is not limited to, 1 to 9 μM, preferably 3 to 7 μM, and more preferably 5 μM.

The Forskolin may be contained in the EM at, but is not limited to, 1 to 20 μM, preferably 5 to 15 μM, and more preferably 10 μM.

The CHIR99021 may be contained in the EM at, but is not limited to, at 1 to 5 μM, preferably 2 to 4 μM, and more preferably 3 μM.

The Matrigel GFR may be contained in the EM at, but is not limited to, 0.2 to 0.8 mg/ml, preferably 0.3 to 0.7 mg/ml, and more preferably 0.5 mg/ml.

The interval between passages of the subculture may vary slightly depending on a seeding density, but may be from 3 to 8 days, preferably from 4 to 7 days, and more preferably from 5 to 6 days, and a medium may be replaced every 2 to 3 days. In addition, a split ratio may be 1:4 to 1:10, preferably 1:5 to 1:10, and more preferably 1:6 to 1:10.

In the present disclosure, after the step (iii), the method may further include: cryopreserving and thawing the hepatic endoderm organoids.

The step of cryopreserving may include a process of leaving the medium on ice for 10 to 30 minutes to separate the Matrigel from the organoids in the medium, and spinning down at 800 to 1,200 rpm for 30 seconds to 1 minute to remove the supernatant. The step of cryopreserving may further include: removing the supernatant, adding Dulbecco's phosphate-buffered saline (DPBS) to loosen the pellet, spinning down at 800 to 1,200 rpm for 30 seconds to 2 minutes to remove the supernatant once again, and adding 0.5 to 2 ml of enzyme to loosen the pellet. The enzyme may be TrypLE Express, Accutase, or 0.25% trypsin-EDTA, preferably TrypLE Express, and more preferably 1 ml of TrypLE Express. The step of cryopreserving may include a process of adding the enzyme to loosen the pellet, leaving it in a water bath for 1 to 3 minutes, pipetting again to loosen all the pellets, adding organoid basal medium (OB medium) containing 10 mM HEPES with Advanced DMEM/F12, 1% GlutaMAX, 100 U/ml penicillin-streptomycin, and 0.1% BSA, and spinning down at 800 to 1,200 rpm for 2 to 4 minutes to remove the supernatant.

In the present disclosure, the matured hepatic organoids may express cytochrome P450 (CYP450) at a high level, and the cytochrome P450 may be labelled with a fluorescent protein.

Specifically, the hepatic organoids may express cytochrome P450 at a higher level than hepatic organoids differentiated by a conventional method in a medium containing extracellular matrix without the use of a bioreactor.

In the present disclosure, the “cytochrome P450 (CYP450)” is a superfamily group of enzymes with heme as a prosthetic group, and is known as a representative catalytic enzyme that performs oxidative metabolism for various exogenous substances such as most drugs or environmental substances, or endogenous substances such as steroids, lipids, etc. The main function of CYP450 enzymes may be said to be mono-oxygenation (or mixed-function oxidase reactions) on various substrates, which requires oxygen molecules and reducing substances of NADPH. One atom of the oxygen molecule binds to the substrate being oxidized and the other atom is reduced to water. In the microsomal CYP450 system, which abundantly contained in hepatocytes, etc., CYP450 enzymes are located on the membrane of the endoplasmic reticulum and receive electrons from NADPH-P450 reductase present together in the membrane to carry out oxygenation. The cytochrome P450 may be one or more selected from the group consisting of CYP1A1, CYP1A2, CYP2A6, CYP2B6, CYP2C8, CYP2C9, CYP2C19, CYP2D6, CYP3A4, and CYP3A5, preferably CYP1A1, CYP1B1, or CYP1A2, and more preferably CYP1A1.

The hepatic organoids may have increased expression levels of major hepatic marker genes, albumin (ALB), I-1 antitrypsin (AAT), hepatocyte nuclear factor 4 alpha (HNF4A), tryptophan 2,3-dioxygenase (TDO2), glucose-6-phosphate (G6P), asialoglycoprotein receptor 1 (ASGR1), cytokeratin 7 (CK7), or multidrug resistance-associated protein 2 (MRP2), and have increased expression levels of CYP450 genes related to drug metabolism, CYP3A4, CYP2C9, CYP2C19, CYP2E1, CYP2D6, CYP2B6, CYP2C8, or CYP1A1, and have increased expression levels of the drug transporter genes, multidrug resistance 1 (MDR1), multidrug resistance-associated protein 2 (MRP2), breast cancer resistance protein (BCRP), or bile salt export pump (BSEP).

In the present disclosure, the “fluorescent protein” refers to a protein that fluoresces, and operates on the principle that general molecules fluoresce by releasing energy in the form of light when they release absorbed energy and return to their ground state.

The fluorescent protein may be one or more selected from the group consisting of, but not limited to, mCherry, green fluorescent protein (GFP), enhanced green fluorescent protein (eGFP), enhanced yellow fluorescent protein (EYFP), cyan fluorescent protein (CFP), mPlum, tdTomato, mStrawberry, J-Red, DsRed, mOrange, mKO, mScarlet, mKate, mBanana, mStrawberry, mCitrine, Venus, YPet, Emerald, CyPet, Cerulean, and T-Sapphire, and preferably, mCherry.

In the present disclosure, the “human pluripotent stem cell line expressing cytochrome P450 fused to a fluorescent protein” may be produced by transfection with a vector comprising a guide RNA targeting a sequence represented by SEQ ID Nos: 1 to 3. The “Guide RNA” is a short single-stranded RNA that specific to a target DNA among the sequences encoding a target gene, and refers to ribonucleic acid that binds complementarily, in whole or in part, to a target DNA sequence and serves to guide an endonuclease protein to the target DNA sequence. The CRISPR/CAS9 system, which is composed of a guide RNA and a Cas9 protein that is responsible for cleaving specific sequences, is a simple and easy way to induce mutations at specific genomic loci and may inhibit the function of specific genes in cells or animals. In other words, when the guide RNA recognizes the target gene, the Cas9 protein binds to the guide RNA and acts as a nuclease to recognize and cleave two guanine bases (GG) located about 3 bp downstream of the target gene, thereby causing a DNA double-strand break (DSB).

In the present disclosure, the vector may further express a Cas9 protein. The “Cas9 protein” is a main protein component of the CRISPR/Cas system, and is protein that may act as an active endonuclease. The Cas9 protein induces double-stranded DNA breaks. In order for the Cas9 protein to accurately bind to the target DNA sequence and cleave the DNA strand, a short sequence of three bases known as a protospacer adjacent motif (PAM) should be present next to the target DNA sequence, and the Cas9 protein extrapolates and cleaves between the third and fourth base pair from the PAM sequence (NGG).

Cas9 protein or gene information may be obtained from publicly available databases such as the National Center for Biotechnology Information (NCBI) GenBank. For example, the Cas9 protein may be one or more selected from the group consisting of, but not limited to, a Cas9 protein derived from Streptococcus pyogenes, a Cas9 protein derived from Campylobacter jejuni, a Cas9 protein derived from Streptococcus thermophilus, a Cas9 protein derived from derived Staphylococcus aureus, and Cas9 protein derived from Neisseria meningitidis, and preferably, a Cas9 protein derived from Streptococcus pyogenes.

In the present disclosure, as a basic framework of the vector, one vector framework selected from the group consisting of pX330, pHAtC, pYLCRISPR/Cas9Pubi, pYLCRISPR/Cas9P35s, pUB-Cas9, pCAMBIA1300DM_OsU6_AarI_Cas9, pCAMBIA1300-OsU3(AarI)-Cas9, Binary vector pUB-Cas9-@GL1, Binary vector pUB-Cas9-@BAR, pYLsgRNA-OsU3, pYLsgRNA-OsU6c, pYLsgRNA-OsU6b, and pYLsgRNA-OsU6a, preferably, the pX330 vector framework, may be used. However, any vector framework known in the art is not particularly limited as long as it successfully expresses the guide RNA and Cas9 protein of the present disclosure to enable transformation of human pluripotent stem cell lines by the CRISPR/Cas9 system.

In the present disclosure, the “transfection” may be carried out by the delivery to the cell line by various methods, such as, but not limited to, microinjection, electroporation, DEAE-dextran treatment, lipofection, nanoparticle-mediated transfusion, protein delivery domain-mediated introduction, and PEG-mediated transfusion, and specifically, in the present disclosure, by electroporation.

In the present disclosure, the method for screening an AHR modulator or a hazardous chemical may further include: (c) selecting a test substance with an altered signal intensity of the fluorescent protein compared to a control that was not administered the test substance. Step (c) may include: determining that the test substance is an AHR agonist or a hazardous chemical if the signal intensity of the fluorescent protein indicates an increased level compared to a control that was not administered the test substance.

In the present disclosure, the screening method may be carried out while the cells are alive, but is not limited thereto.

In the present disclosure, the “aryl hydrocarbon receptor (AHR)” is known as a major nuclear receptor that regulates xenobiotic metabolism in the liver, and has recently been reported to be associated with various metabolic diseases through AHR genetically modified mouse models. In the present disclosure, the “AHR modulator” may comprise an AHR agonist or an AHR antagonist. In the present disclosure, the “AHR agonist” may refer to a substance that activates the action of an AHR receptor, and the “AHR antagonist” may refer to a substance that inhibits the action of an AHR receptor.

In the present disclosure, the “hazardous chemicals” refer to toxic substances, permitted substances, restricted or prohibited substances, accident prevention substances, or any other chemicals that are or are likely to be hazardous or harmful, and may comprise carbon dioxide, volatile organic compounds, carbon monoxide, etc., and chemicals produced by molds, fine dust, etc. In the present disclosure, the hazardous chemicals are not limited in type as long as they are hazardous or harmful that may be exposed in daily life, and are not limited to chemicals that are correlated with AHR. Specifically, the hazardous chemicals may induce toxicity in human body by inducing the expression of CYP1A1 through binding to AHR and metabolizing it, and may induce toxicity in human body by indirectly inducing the expression of CYP1A1 without binding to the AHR.

In the present disclosure, the method for screening an AHR modulator or a hazardous chemical may select an AHR modulator or a hazardous chemical with higher sensitivity, compared to a method for screening an AHR modulator or a hazardous chemical by treating hepatocytes derived from a human pluripotent stem cell line expressing cytochrome P450 fused to a fluorescent protein, with a test substance.

In an aspect for achieving the above object, the present disclosure provides a composition for screening an AHR modulator or a hazardous chemical, comprising hepatic organoids derived from human pluripotent stem cell lines expressing cytochrome P450 fused to a fluorescent protein.

The “fluorescent proteins”, “cytochrome P450”, “human pluripotent stem cells”, “hepatic organoids”, “AHR modulators”, and “hazardous chemicals” are as defined above.

Hereinafter, the present disclosure will be described in more detail with reference to examples. These examples are merely for illustrating the present disclosure, and it will be apparent to those skilled in the art that the scope of the present disclosure is not to be construed as being limited by these examples.

### BRIEF DESCRIPTION OF DRAWINGS

The above and other aspects, features, and advantages of the present disclosure will be more clearly understood from the following detailed description taken in conjunction with the accompanying drawings, in which:

FIG. 1 shows a schematic diagram of the targeting vector used to generate a CYP1A1-mCherry human pluripotent stem cell line (hPSC).

FIG. 2 shows target sites of three sgRNAs used to generate CYP1A1-mCherry human pluripotent stem cell lines (hPSCs).

FIG. 3 shows a schematic process of a process of producing hepatic endoderm organoids (HEOs) from a CYP1A1-mCherry human pluripotent stem cell line.

FIG. 4 shows a result obtained by observing cellular changes in a process of producing hepatic endoderm organoids (HEOs) from a CYP1A1-mCherry human pluripotent stem cell line and confirming whether or not hepatic endoderm organoid-related markers EpCAM, SOX9, and CK19 are expressed.

FIG. 5 shows a schematic process of the differentiation from hepatic endoderm organoids (HEOs) to hepatic organoids (HOs).

FIG. 6A shows a result obtained by confirming whether or not albumin (ALB), AAT, HNF4A, CK7, and MRP2 are expressed in hepatic organoids (HOs) derived from CYP1A1-mCherry human pluripotent stem cell lines prepared in the present disclosure.

FIG. 6B shows comparison results between mRNA expression levels of major hepatic factors, albumin (ALB), HNF4A, AAT, TDO2, ASGR1, and G6P of hepatic organoids (HOs) derived from CYP1A1-mCherry human pluripotent stem cell lines prepared in the present disclosure, and hepatic endoderm organoids (HEOs).

FIG. 6C shows comparison results between expression levels of cytochrome P450 (CYP450) genes and drug transporter genes related to drug metabolism of hepatic organoids (HOs) derived from CYP1A1-mCherry human pluripotent stem cell lines prepared in the present disclosure, and hepatic endoderm organoids (HEOs).

FIG. 7A shows a result of measuring the mCherry fluorescence intensity and the CYP1A1 mRNA expression levels after confirming the degree of increase in CYP1A1 fluorescence expression through an imaging device when the hepatic organoids derived from CYP1A1-mCherry human pluripotent stem cell lines prepared in the present disclosure were treated with AHR agonist drugs TCDD and BaP.

FIG. 7B shows a result obtained by confirming the degree of increase in CYP1A1 fluorescence expression over time when the hepatic organoids derived from CYP1A1-mCherry human pluripotent stem cell lines prepared in the present disclosure were treated with AHR agonist drugs, TCDD and BaP.

FIGS. 8A and 8B show the information on 17 chemicals that were confirmed to increase mCherry fluorescence intensity in hepatocytes derived from a CYP1A1 fluorescently labeled stem cell line, and a result obtained by confirming an increase or decrease in real-time fluorescence intensity after treating the hepatic organoids derived from CYP1A1-mCherry human pluripotent stem cell lines according to the present disclosure with 17 chemicals for 24 hours.

FIG. 9A shows comparison results between the degree of increase in CYP1A1 fluorescence expression when hepatic organoids derived from CYP1A1-mCherry human pluripotent stem cell lines prepared in the present disclosure were treated with seventeen chemicals, and when hepatocytes (hiPSC-Hep) derived from CYP1A1 fluorescently labeled stem cell lines were treated seventeen chemicals.

FIG. 9B shows the results obtained by observing the change in in real-time fluorescence intensity observed after the hepatic organoids of the present disclosure with seven chemicals that were shown to increase mCherry fluorescence intensity.

FIGS. 10A and 10B show the results obtained by confirming the change in fluorescence intensity, and the change in CYP1A1 mRNA expression levels when the hepatic organoids derived from CYP1A1-mCherry human pluripotent stem cell lines prepared in the present disclosure was treated together with an AHR agonist (TCDD, BaP) and an antagonist (CH223191).

FIG. 11 shows a list of six substances that were confirmed to lower mCherry fluorescence intensity in hepatocytes derived from a CYP1A1 fluorescently labeled stem cell line.

FIG. 12 shows mCherry fluorescence intensity observed in real-time when the hepatic organoids derived from CYP1A1-mCherry human pluripotent stem cell lines prepared in the present disclosure were treated with six chemicals confirmed to lower mCherry fluorescence intensity in hepatocytes derived from a CYP1A1 fluorescently labeled stem cell line.

FIGS. 13A-13F show the results obtained by confirming a cell viability when the hepatic organoids derived from CYP1A1-mCherry human pluripotent stem cell lines prepared in the present disclosure were treated with thirty substances selected from hazardous chemicals.

FIGS. 14A and 14B show the change in mCherry fluorescence intensity observed in real-time when the hepatic organoids derived from CYP1A1-mCherry human pluripotent stem cell lines prepared in the present disclosure were treated with thirty hazardous chemicals.

FIGS. 15A and 15B show the change in fluorescence intensity observed in real-time by treating the hepatic organoids derived from CYP1A1-mCherry human pluripotent stem cell lines prepared in the present disclosure with three chemicals, tBHQ, 1,2-NQ, and 1,4-NQ that were shown to increase mCherry fluorescence intensity, at different concentrations.

FIGS. 16A and 16B show the results obtained by confirming a decrease in mCherry fluorescence intensity and a decrease in CYP1A1 gene expression levels when three chemicals, tBHQ, 1,2-NQ, and 1,4-NQ that were shown to increase mCherry fluorescence intensity, were treated together with CH223191, an AHR antagonist.

### DETAILED DESCRIPTION

Hereinafter, the present disclosure will be described in more detail with reference to examples. However, these examples are intended to illustrate the present disclosure and the scope of the present disclosure is not limited to these examples.

### Example 1. Preparation of Hepatic Organoids Derived from CYP1A1 Fluorescently Labelled Human Pluripotent Stem Cell Lines

In order to create a model for screening an aryl hydrocarbon receptor (AHR) modulator or a hazardous chemical, hepatic organoids were prepared from a CYP1A1 fluorescently labelled human induced pluripotent stem cell line using the following method.

### 1.1. Preparation of CYP1A1 Fluorescently Labelled Human Pluripotent Stem Cell Line

### 1.1.1. Human Pluripotent Stem Cell Culture

First, human pluripotent stem cells derived from human dermal fibroblasts were maintained in mTeSR-E8 medium containing Vitronectin XF™ (Stem Cell Technologies, Vancouver, Canada) and dissociated every 3 or 4 days using 0.5 mM EDTA in Dulbecco's Phosphate-Buffered Saline (DPBS).

### 1.1.2. Preparation of mCherry Fluorescently Labelled Human Pluripotent Stem Cell Lines Using CRISPR-Cas9 System

### (1) Construction of CYP1A1 Targeting Vectors

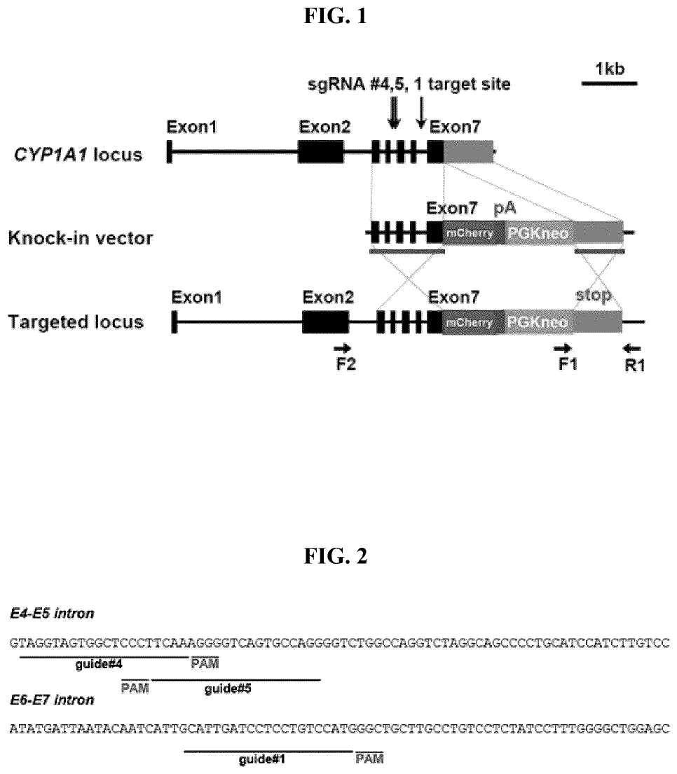

A schematic diagram of the targeting vector used to prepare a CYP1A1-mCherry human pluripotent stem cell line (hPSC) is shown in FIG. 1.

Specifically, a human codon-optimised SpCas9 and chimeric guide RNA expression plasmid (pX330-U6-Chimeric_BB-CBh-hSpCas9; Addgene, Watertown, MA, USA) were used to target vector construction. Single guide RNAs (sgRNAs) were constructed by using an online CRISPR design tool (http://crispr.mit.edu) in the intronic region of the target gene, and sgRNAs were selected by using the lowest off-target effect.

Three single-guided RNAs (sgRNAs) were designated to target either the exon 4-exon 5 (E4-E5) or exon 6-exon 7 (E6-E7) introns, the target sequences of the three sgRNAs are shown in Table 1 below, and the target sites of the three sgRNAs used are shown in FIG. 2. Subsequent experiments were carried out according to the target sequence cloning protocol of Zhang laboratory.

TABLE 1

| | | | | PCR | | SEQ |
|---|---|---|---|---|---|---|
| | | | Target | analysis | | ID |
| Locus | sgRNA | Score | site | colony | Freeze | No |
| E6-E7 | Guide #1 | 78 | GCATTG | 6/13 | 5 | 1 |
| intron | | | ATCCTC | | | |
| | | | CTGTCC | | | |
| | | | AT | | | |
| E4-E5 | Guide | 73 | TAGGTA | 12/29 | 9 | 2 |
| intron | #4-2 | | GTGGCT | | | |
| | | | CCCTTC | | | |
| | | | AA | | | |
| | Guide | 73 | CTGGCA | 4/9 | 3 | 3 |
| | #5 | | CTGACC | | | |
| | Reverse | | CCTTTG | | | |
| | | | AA | | | |

Meanwhile, the pMCDT-A vector (Addgene, plasmid #27179) was used as the framework of the targeting vector. The human CYP1A1 gene was amplified by polymerase chain reaction (PCR) using genomic DNA extracted from hPSCs. The 5′ and 3′ homologous arms were targeted 1.2 kb upstream and 0.93 kb downstream of the stop codon in CYP1A1, respectively. mCherry and PGKneo were amplified by PCR from plasmids containing them. After sequence verification, each fragment was ligated using T4 DNA ligase (Promega, Madison, WI, USA). 5′ homologous arms, mCherry, PGKneo, and 3′ homologous arms were ligated using SmaI, HindIII, and SalI, respectively. The resulting final fragment was inserted into the pMCDT-A vector using NotI and XhoI to construct the targeting vector.

### (2) Transfection

For transfection, hPSCs at passage 28 were prepared as single cells (2.5×10⁷ cells/ml) by dissociation with 0.5 mM EDTA in DPBS. 4 g of targeting vector and 1 g of Cas9 vector were added to a cell suspension and electroporated under 1,600 V, 20 ms, 1 pulse conditions using a NEON® (Invitrogen, Carlsbad, CA, USA) transfection system. After electroporation, cells were seeded in Matrigel®-coated 48-well plates at 1,000 viable cells per well. Cells were expanded for 48 h and then selected with 50 g/ml geneticin (G418 sulfate, Gibco) for 2 weeks. Then, drug-resistant clones were manually selected to expand single-cell clone, and clones of a human pluripotent stem cell line expressing mCherry-fused CYP1A1 were selected.

For subsequent hepatic organoids production, CYP1A1-mCherry hPSCs deposited at the Biological Resource Center of the Korea Research Institute of Bioscience and Biotechnology under accession number KCTC 14186BP were used at passages 29 to 40.

### 1.2. Hepatic Endoderm Organoids Production

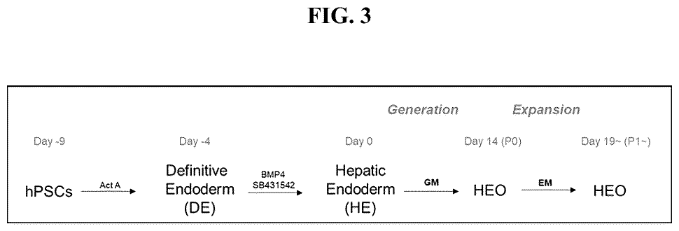

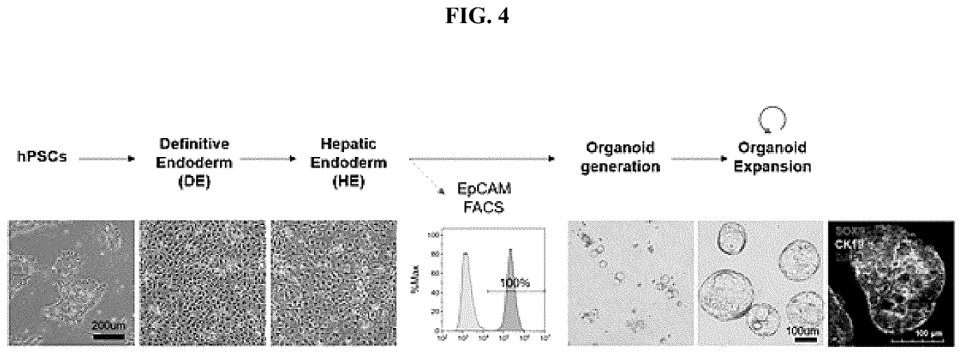

The schematic process of producing Hepatic Endoderm Organoids (HEOs) from the CYP1A1-mCherry human pluripotent stem cell line selected in 1.1 above is shown in FIG. 3.

### 1.2.1. CYP1A1-mCherry Human Pluripotent Stem Cell (hPSC) Seeding: Day −10

First, CYP1A1-mCherry human pluripotent stem cell lines (hPSCs) were seeded on Day—10 based on the time of hepatic endoderm differentiation (Day 0). As CYP1A1-mCherry human pluripotent stem cell culture medium, TeSR™-E8™ (Stem Cell Technologies) was used.

Specifically, a 12-well culture plate for seeding was prepared, and coating was carried out at 37° C. for 30 minutes by adding 0.5 ml of diluted Matrigel per well. The Matrigel was used by diluting its stock solution with hESC qualified Matrigel (Corning, Cat No. 354277) according to the Certificate of Analysis (COA). During the coating of the Matrigel, the cultured hPSCs were taken out of the incubator and the medium was removed.

Then, 1 ml of Dulbecco's phosphate-buffered saline (DPBS) (containing no Ca²⁺ and Mg²⁺) medium containing 0.5 mM EDTA was added and cultured at 37° C. for 4 to 5 minutes. After carefully removing the cells from the incubator, the plate was tilted to suck up the EDTA. 1 ml of 12 ml of the prepared TeSR™-E8™ with 2 μM Y-27632 was used to harvest the cells. The harvested cells were added to a conical tube containing 11 ml of TeSR™-E8™ with 2 μM Y-27632 and mixed by gentle pipetting. The Matrigel-coated plate was taken out of the incubator and all of the Matrigel was removed by suction. Then, the recovered cells were dispensed at 1 ml per well and cultured in an incubator at 37° C. and 5% CO₂ for 1 day.

### 1.2.2. Differentiation of CYP1A1-mCherry Human Pluripotent Stem Cells (hPSCs) into Definitive Endoderm (DE) Cells: Day −9 to Day −4

Definitive Endoderm (DE) differentiation medium 1 with the composition as shown in Table 2 below was prepared in 12 ml and left at 37° C. The hPSCs cultured for 1 day were removed from the incubator and the medium was removed. The prepared endoderm differentiation medium 1 was added at 1 ml per well and cultured in an incubator at 37° C. and 5% CO₂ for 24 hours.

TABLE 2

| Name | Composition |
|---|---|
| Endoderm | RPMI-1640 containing 0.1% BSA and |
| differentiation | 1% B27 supplement |
| medium 1 | 50 ng/ml Activin A (ActA) |
| (DE medium 1) | 0.5 mM Sodium butyrate |
| | 3 μM CHIR99021 |

After 24 hours, the medium was replaced with endoderm differentiation medium 2 (DE medium 2) with the composition as shown in Table 3 below and cultured in an incubator at 37° C. and 5% CO₂ for 4 days, and the medium was replaced every 24 hours.

TABLE 3

| Name | Composition |
|---|---|
| Endoderm | RPMI-1640 containing 0.1% BSA and |
| differentiation | 1% B27 supplement |
| medium 2 | 50 ng/ml Activin A (ActA) |
| (DE medium 2) | 0.1 mM Sodium butyrate |

### 1.2.3. Differentiation of Definitive Endoderm Cells into Hepatic Endoderm Cells: Day −4 to Day 0

12 ml of Hepatic Endoderm (HE) differentiation medium with the composition as shown in Table 4 below was prepared, left at 37° C., and the cells differentiated into endoderm were taken out of the incubator and the medium was removed. The prepared hepatic endoderm differentiation medium was added at 1 ml per well and cultured in an incubator at 37° C. and 5% CO₂ for 4 days, and the medium was replaced every 24 hours.

TABLE 4

| Name | Composition |
|---|---|
| Hepatic endoderm | RPMI-1640 containing 0.1% BSA and |
| differentiation | 1% B27 supplement |
| medium | 50 ng/ml BMP4 |
| (HE medium) | 10 μM SB431542 |

After completion of differentiation, the Epithelial cell adhesion molecule (EpCAM) positive population of some cells were analyzed, and if the EpCAM-positive cells were 95% or more, it proceeded to the organoids production stage. As a result of FACS analysis, it could be confirmed that EpCAM expression was high at 100%, as shown in FIG. 4.

### 1.2.4. Hepatic Endoderm Organoids (HEOs) Production: Day 0 to Day 14

Generation medium (GM) for hepatic endodermal organoid (HEO) with the composition as shown in Table 5 below was prepared at least one day before the experiment.

TABLE 5

| Name | Composition |
|---|---|
| Hepatic endoderm | Advanced DMEM/F12 containing |
| organoid generation | 10 mM HEPES, 1% GlutaMAX, 100 U/ml |
| medium (GM) | penicillin-streptomycin, 0.1% BSA, 1% B27 |
| | supplement minus vitamin A, 1% N2 supplement |
| | 50 ng/ml FGF10 |
| | 25 ng/ml HGF |
| | 10 mM Nicotinamide |
| | 10 nM [Leu¹⁵]-Gastrin I human |
| | 1.25 mM N-acetyl-L-cysteine |
| | 5 μM A83-01 |
| | 10 μM Forskolin |
| | 3 μM CHIR99021 |
| | 0.5 mg/ml Matrigel growth factor reduced (GFR) |

The differentiated hepatic endoderm cells (only 3 or 4 wells in good condition were used) were taken out of the incubator, the medium was removed, and the cells were washed once with DPBS medium without Ca²⁺ and Mg²⁺.

500 ul Accutase was added per well and cultured at 37° C. for 10 minutes. The cells isolated as single cells were transferred to a 15 ml conical tube containing 10 ml of organoid basal (OB) medium, centrifuged at 1,200 rpm for 3 minutes to remove the supernatant.

The cell pellet was resuspended in 1 ml of cold GM and transferred to a 1.5 ml tube. Then, cells were counted and cultured.

### 1.2.5. Hepatic Endoderm Organoids Culture

The GM for hepatic endoderm organoids was pre-dispensed into a 24-well ultra-low attachment plate at 0.5 ml per well. The medium was stored on ice during use, to be always maintained at 4° C. to prevent Matrigel gelation.

10,000 single hepatic endoderm cells were added per well and cultured for 14 days in an incubator at 37° C. and 5% CO₂. The plates were placed in the incubator and the cells were spread evenly to prevent them from growing in clumps before closing the door, and the medium was replaced every 3 days.

Medium replacements were carried out based on 1 well of a 24-well plate, using 1,000 P to recover all the medium containing organoids cultured in suspension, transfer to a 1.5 ml tube, centrifuge at 1,200 rpm for 1 minute, remove the supernatant, resuspend with 0.5 ml of fresh GM for hepatic endoderm organoids (s-HEOs) in suspension, sprinkle back onto the plate, and culture in an incubator at 37° C. and 5% CO₂ for 3 days.

### 1.3. Subculture of Hepatic Endoderm Organoids

The hepatic endoderm organoids were taken out of the incubator. All organoids containing the medium in the wells, were collected into a 15 ml tube using 1,000 P, spun down at 1,000 rpm for 1 minute, and the supernatant was removed.

Then, 1 ml of DPBS was added, the pellet was loosened by pipetting, spun down at 1,000 rpm for 1 minute to remove the supernatant. 1 ml of TrypLE Express was added, the pellet was loosened by pipetting, and left for 10 minutes in an incubator at 37° C. and CO₂. All pellets were loosened by pipetting and then spun down at 1,000 rpm for 3 minutes.

Fresh expansion medium (EM) for HEOs was pre-dispensed into a fresh 24-well ultra-low attachment plate at 0.5 ml per well. The EM was prepared with the same composition as the GM. After removing the supernatant following the above spin-down, 200 L of ice-cold EM was added, the pellet was loosened, and 20 L per well were dispensed and cultured in an incubator at 37° C. and 5% CO₂ for 5 to 6 days. Before placing the plate in the incubator and closing the door, the plate was shaken up and down and side to side to spread the cells evenly to prevent them from growing in clumps. The medium was replaced every three days.

Medium replacements were carried out base on 1 well of a 24-well plate, using 1,000 P to recover all the medium containing organoids in suspension, transfer to a 1.5 ml tube, centrifuge at 5,000 rpm at maximum speed based on a mini-centrifuge, remove the supernatant, resuspend with 0.5 ml of fresh EM for HEOs, sprinkle back onto the plate, and culture in an incubator at 37° C. and 5% CO₂ for 3 days.

The interval between passages of the subculture varies slightly depending on the seeding density, but 5 to 6 days was the optimal interval, and the split ratio was 1:6 to 1:10, but was adjusted depending on the amount of cells.

### 1.4. Characterization of Hepatic Endoderm Organoids

To characterize the hepatic endoderm organoids prepared above, the hepatic endoderm organoids were observed by fluorescent staining, and the expression changes of SRY-box transcription factor 9 (SOX9) and cytokeratin 19 (CK19) were measured as hepatic endoderm organoid (HEO) markers.

As a result, as shown in FIG. 4, it was confirmed that the prepared hepatic endoderm organoids had the properties of hepatic progenitor cells by observing the expression of representative hepatic progenitor cell markers SOX9 and CK19, and subsequent experiments were conducted.

### 1.5. Freezing and Thawing of Hepatic Endoderm Organoids

### 1.5.1. Freezing

The hepatic endoderm organoids were taken out of the incubator. All organoids containing the medium in the wells, were collected into a 1.5 ml tube using 1,000 P and the experiment proceeded with being placed on ice.

In order to separate the Matrigel from the organoids, they were left on ice for 20 minutes, spun down at 5,000 rpm at maximum speed based on a mini-centrifuge, and then the supernatant was removed. Then, 1 ml of DPBS was added, the pellet was loosened by pipetting, spun down at 1,000 rpm for 1 minute to remove the supernatant. 1 ml of TrypLE Express was added, the pellet was loosened by pipetting, and left in a water bath at 37° C. for 2 minutes.

All pellets were loosed by pipetting, 1.5 ml of organoid basal (OB) medium was added, spun down at 1,000 rpm for 3 minutes, and 1 to 1.5 ml of CryoStor CS10 (Stem cell Technologies, cryoprotectant) was added to resuspend. Aliquots of 0.5 ml were dispensed into a cell cryotube, placed in a freezing container, stored in a deep freezer for 1 day, and then transferred to an LN2 tank the following day.

### 1.5.2. Thawing

One cryotube was taken out of the LN2 tank and thawed in a water bath at 37° C. when it was half melted, it was taken out of the water bath and transferred to a 15 ml conical tube containing 10 ml of pre-prepared organoid basal (OB) medium. The composition of the organoid base (OB) medium is as shown in Table 6 below.

TABLE 6

| Name | Composition |
|---|---|
| Organoid base | Advanced DMEM/F12 containing |
| medium | 10 mM HEPES, 1% GlutaMAX, |
| (OB) | 100 U/ml penicillin-streptomycin |

After centrifugation at 1,000 rpm for 3 minutes, the supernatant was removed and the organoids pellet was resuspended with 20 μM Y27632 containing 1 to 1.5 ml cold EM. Then, aliquots of 0.5 ml were dispensed into a fresh 24-well ultra-low attachment plate.

It was cultured in an incubator at 37° C. and 5% CO₂ for 3 to 4 days. Before placing the plate in the incubator and closing the door, the organoids pieces was evenly spread to prevent them from growing in clumps. After 3 to 4 days, the medium was replaced with the EM and cultured.

### 1.6. Hepatic Organoids Differentiation (Maturation)

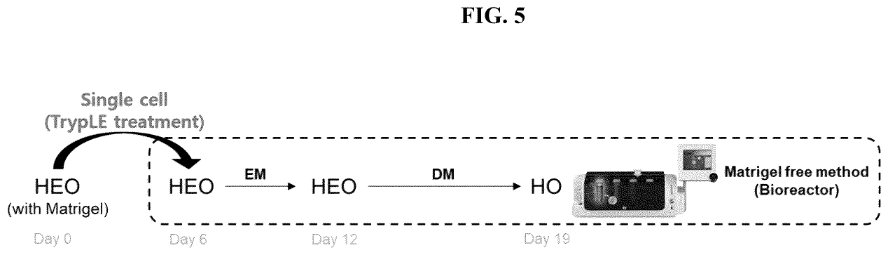

The schematic process of the differentiation from hepatic endoderm organoids (HEOs) to hepatic organoids (HOs) is shown in FIG. 5.

As shown in Example 1.3, HEO subculture was carried out, and on day 6 of subculture, the subculture was treated with TrypLE to make it single cells. Then, 2×10⁶ cells were counted, placed in 20 ml of EM without Matrigel, placed in a bioreactor-specific tube, and cultured at 37° C., 5% CO₂, and 80 rpm for 6 days.

Then, on days 12 to 19 of culture, the medium was replaced with the DM without Matrigel and cultured at 37° C., 5% CO₂, and 60 rpm for 7 days, with medium replacements every 3 days. The composition of the DM is as shown in Table 7 below.

TABLE 7

| Name | Composition |
|---|---|
| Hepatic organoid | Advanced DMEM/F12 containing 10 mM HEPES, |
| differentiation | 1% GlutaMAX, 100 U/ml penicillin-streptomycin, |
| medium (DM) | 0.1% BSA, 1% B27 supplement minus vitamin A |
| | 25 ng/ml BMP7 |
| | 100 ng/ml FGF19 |
| | 25 ng/ml HGF |
| | 1.25 mM N-acetyl-L-cysteine |
| | 0.5 μM A83-01 |
| | 10 μM DAPT |
| | 3 μM Dexamethasone |
| | 0.5 mg/ml Matrigel GFR |

A medium replacement method was to leave the tube where organoids were cultured in the bioreactor on a clean bench for 1 minute based on one tube, remove the supernatant except for the settled organoids, and then replace the medium with a medium without Matrigel. Then, the medium was replaced by capping the tubes and placing them back into a bioreactor.

On day 19, the differentiation into hepatic organoids was completed, and the organoids obtained at this point were subjected to a screening process for the properties of hepatic organoids and AHR regulators and hazardous chemicals using the organoids.

### Example 2. Characterization of Hepatic Organoids

According to Example 1 above, CYP1A1 fluorescently labelled human pluripotent stem cell line was differentiated into hepatic organoids, and changes in the expression of main hepatic factors were measured to characterize the differentiated hepatic organoids.

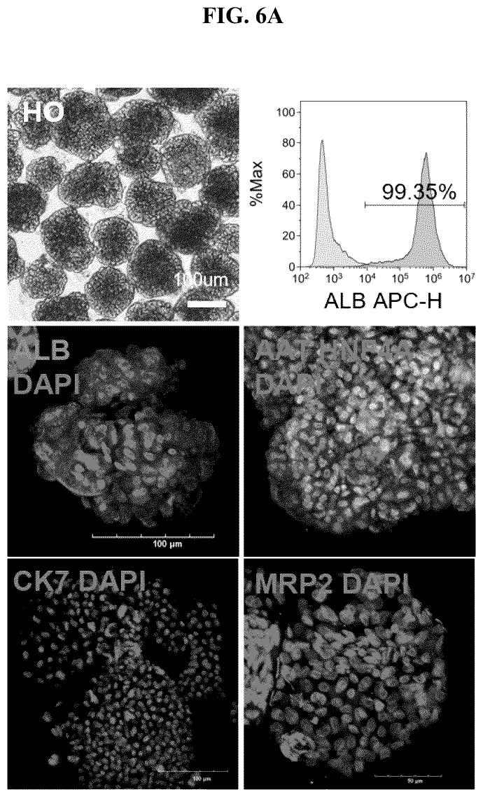

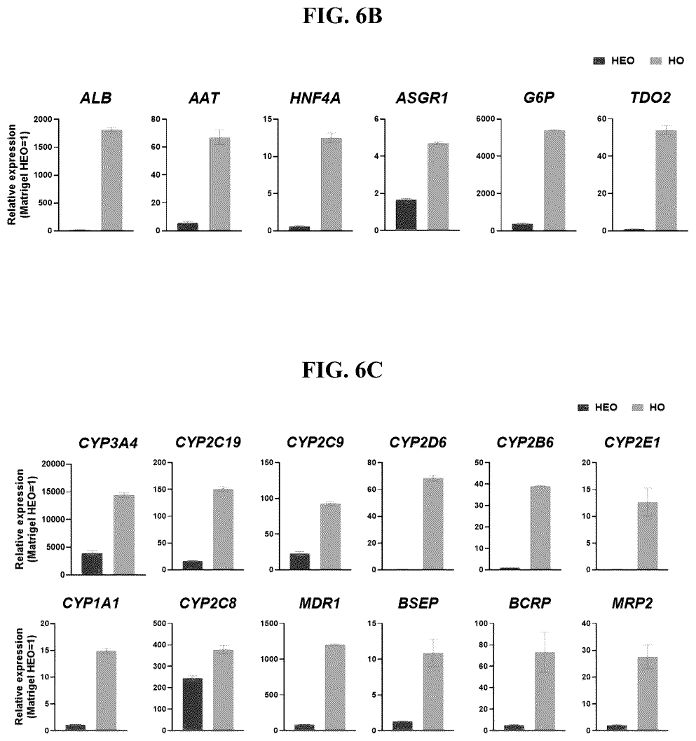

As a result of observing the hepatic organoids by fluorescent staining, as shown in FIG. 6A, it was confirmed that albumin (ALB), alpha-1 antitrypsin (AAT), hepatocyte nuclear factor 4 alpha (HNF4A), cytokeratin 7 (CK7), and multi-drug resistance-associated protein 2 (MRP2) were well expressed as markers in the hepatic organoids (HOs). As a result of FACS, it could be confirmed that albumin expression was high at 99.35%.

Furthermore, as a result of measuring the mRNA expression level of major hepatic factors in differentiated liver organoids, as shown in FIG. 6B, it was confirmed that the expression of hepatocyte markers ALB, AAT, HNF4A, asialoglycoprotein receptor 1 (ASGR1), glucose-6-phosphate (G6P), and tryptophan 2,3-dioxygenase (TDO2) was increased that that of hepatic endoderm organoids (HEOs).

In addition, as shown in FIG. 6C, it was confirmed that the mRNA expression levels of cytochrome P450 (CYP450) genes related to drug metabolism, CYP3A4, CYP2C19, CYP2C9, CYP2D6, CYP2B6, CYP2E1, CYP1A1, and CYP2C8, and drug transporter genes, multidrug resistance 1 (MDR1), bile salt export pump (BSEP), breast cancer resistance protein (BCRP), and multidrug resistance-associated protein 2 (MRP2) were all increased compared to hepatic endoderm organoids (HEOs).

### Example 3. Confirmation of Effect of AHR Modulator Dru2 Screening Using CYP1A1 Fluorescently Labelled Human Pluripotent Stem Cell Lines-Derived Hepatic Organoids

The CYP1A1 fluorescently labelled human pluripotent stem cell line-derived hepatic organoids in prepared in Example 1 were treated with dioxin (2,3,7,8-tetrachlorodibenzo-p-dioxin, TCDD) and benzo[a]pyrene (BaP), which are AHR agonist drugs that bind to the AHR to be activated, and 24 hours later, an increase in CYP1A1 fluorescence expression was confirmed through an imaging device and CYP1A1 mRNA expression level was measured.

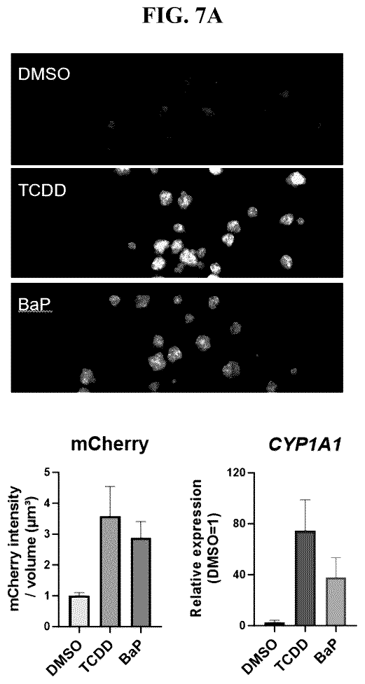

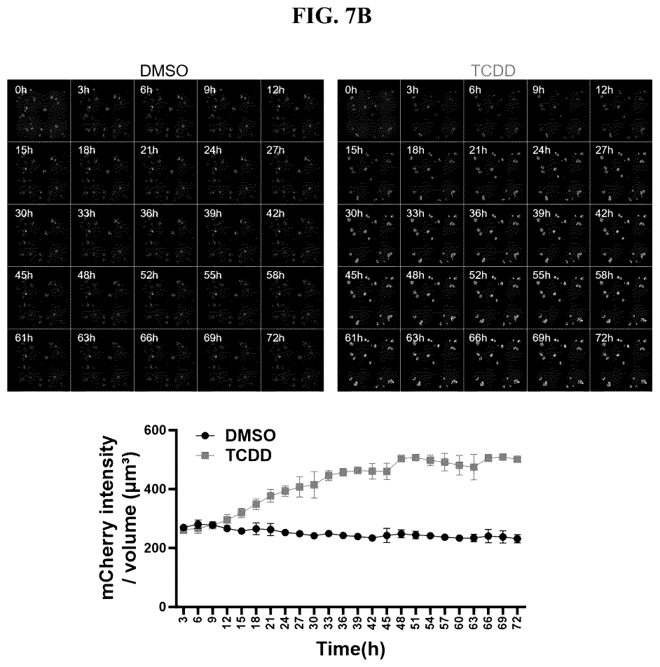

As a result, as shown in FIG. 7A, it was confirmed that the degree of increase in mCherry fluorescence according to TCDD and BaP treatment was consistent with the result of increased mRNA expression leavle of CYP1A1. As shown in FIG. 7B, it was confirmed that the mCherry fluorescence intensity was increased in a time-dependent manner when real-time mCherry fluorescence imaging was observed by time-lapse imaging at 3-hour intervals up to 72 hours after TCDD treatment of hepatic organoids.

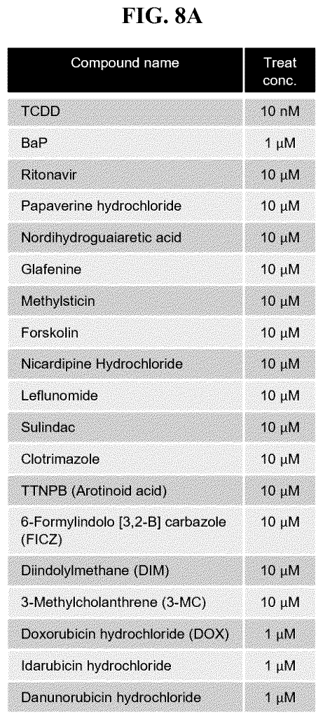

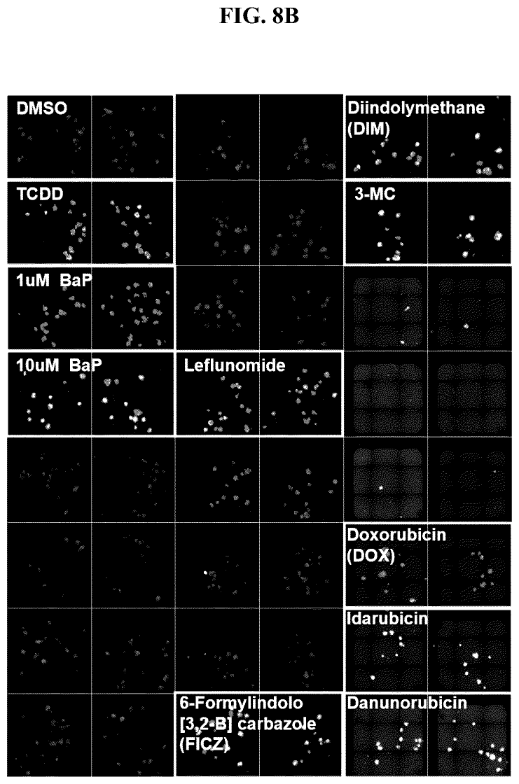

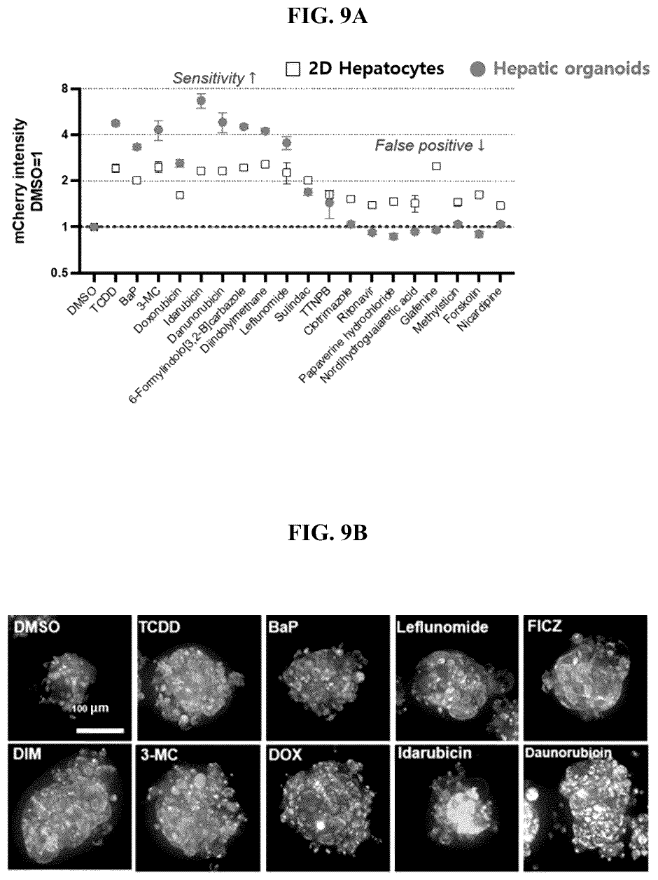

In addition, the hepatic organoids derived from CYP1A1 fluorescently labelled stem cell lines according to the present disclosure were treated with the seventeen chemicals of FIG. 8A, which were found to increase mCherry fluorescence intensity by 30% in the conventional CYP1A1 fluorescently labelled stem cell line-derived hepatocytes. The results of observing the increase and decrease of real-time fluorescence after 24 hours of treatment with the chemicals are shown in FIG. 8B. As shown in FIG. 9A, it was confirmed that the intensity of mCherry fluorescence was increased in the CYP1A1 fluorescently labelled stem cell line-derived hepatic organoids (“Hepatic Organoids”) according to the present disclosure compared to the conventional CYP1A1 fluorescently labelled stem cell line-derived hepatocytes (“hiPSC-Hep”). Results observing real-time fluorescence of seven chemicals which increase mCherry fluorescence intensity in hepatic organoids: leflunomide, 6-formylindolo [3,2-B]carbazole (FICZ), diindolylmethane (DIM), 3-methylcholanthrene (3-MC), doxorubicin (DOX), idarubicin, and daunorubicin, are shown in FIG. 9B.

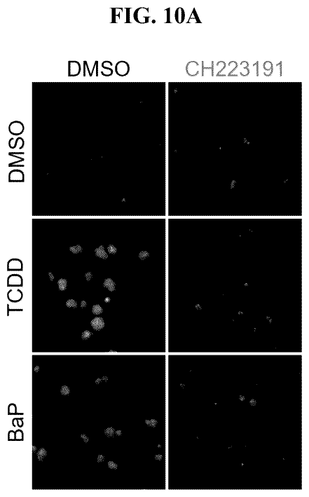

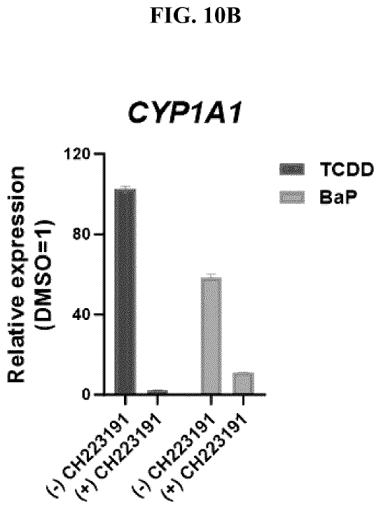

Meanwhile, when an AHR agonist drugs TCDD and BaP, which have been shown to increase mCherry fluorescence in the CYP1A1 fluorescently labelled stem cell line-derived hepatic organoids according to the present disclosure, were treated with CH223191 known as an AHR antagonist, the fluorescence intensity and CYP1A1 gene expression level were decreased, as shown in FIGS. 10A and 10B.

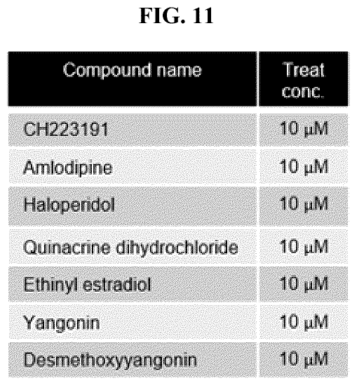

Therefore, the CYP1A1 fluorescently labelled stem cell line-derived hepatic organoids were treated with the six chemicals in FIG. 11, which have been shown to reduce mCherry fluorescence intensity by 70% in the conventional CYP1A1 fluorescently labelled stem cell line-derived hepatocytes, and the change in mCherry fluorescence intensity was observed in real-time.

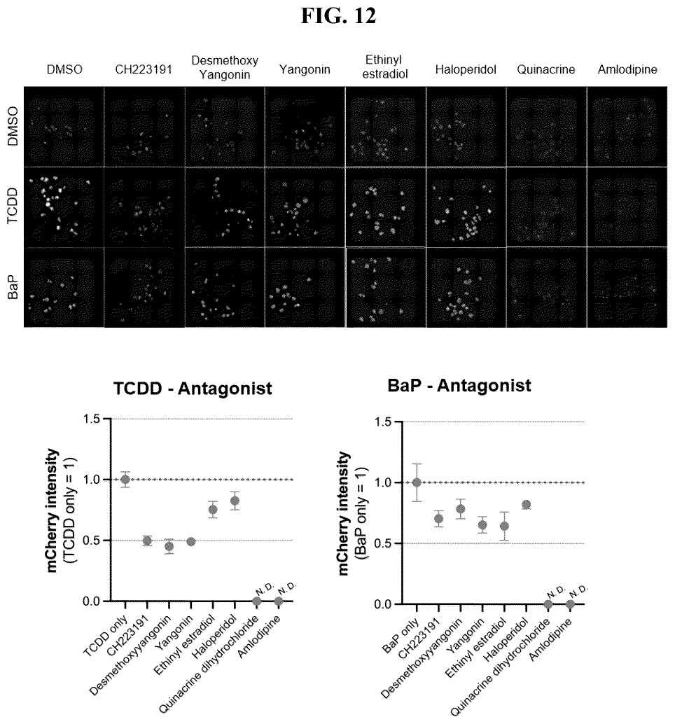

As a result, as shown in FIG. 12, it was confirmed that the mCherry fluorescence intensity was decreased when the six substances were treated together with TCDD and BaP.

From the above results, it is considered that the AHR modulator screening method using the CYP1A1 fluorescently labelled stem cell line-derived hepatic organoids prepared in the present disclosure can effectively screen AHR agonist drugs that enhance AHR activity, or AHR antagonist drugs that reduce AHR activity, and has a higher sensitivity in screening AHR modulator drugs than the conventional screening method using the CYP1A1 fluorescently labelled stem cell line-derived hepatocytes, and thus can screen out false positives.

### Example 4. Confirmation of Effect of Screening Hazardous Chemicals Using CYP1A1 Fluorescently Labelled Human Pluripotent Stem Cell Line-Derived Hepatic Organoids

To further explore the hazardous chemicals that induce CYP1A1 regulation using the CYP1A1 fluorescently labelled human pluripotent stem cell line-derived hepatic organoids, thirty hazardous chemicals were selected from those reported to the National Institute of Environmental Research and are shown in Table 8 below.

TABLE 8

| NO. | Hazardous chemical names |
|---|---|
| 1 | N,N-Dimethylformamide |
| 2 | Resorcinol |
| 3 | Imidazolidinethione |
| 4 | Acrylamide |
| 5 | Triphenyl phosphite |
| 6 | N-Metylformamide |
| 7 | Tetramethylammonium |
| 8 | 2-Etoxyethanol |
| 9 | Benzyl butyl phthalate |
| 10 | Quinoline |
| 11 | Chloropyridine |
| 12 | Dinitrotoluene |
| 13 | Chlorobenzophenone |
| 14 | 3-Chloro-5-(trifluoromethyl)-2-pyridyl]amine; |
| | ACTP |
| 15 | Dibutyl phthalate |
| 16 | Nitrophenol |
| 17 | Aniline |
| 18 | 2-Hydroxyethyl acrylate |
| 19 | tert-Butylhydroquinone; tBHQ |
| 20 | 1-Nitronaphathalene |
| 21 | Acrylic acid |
| 22 | Bisphenol A |
| 23 | Diisobutyl phthalate |
| 24 | Tributyltin chloride |
| 25 | 1,2-Naphtoquinone |
| 26 | 1,4-Naphtoquinone |
| 27 | 2-Nitrotoluene |
| 28 | Diaminodiphenylmethane |
| 29 | p-Cresol |
| 30 | 5-Fluorouracil |

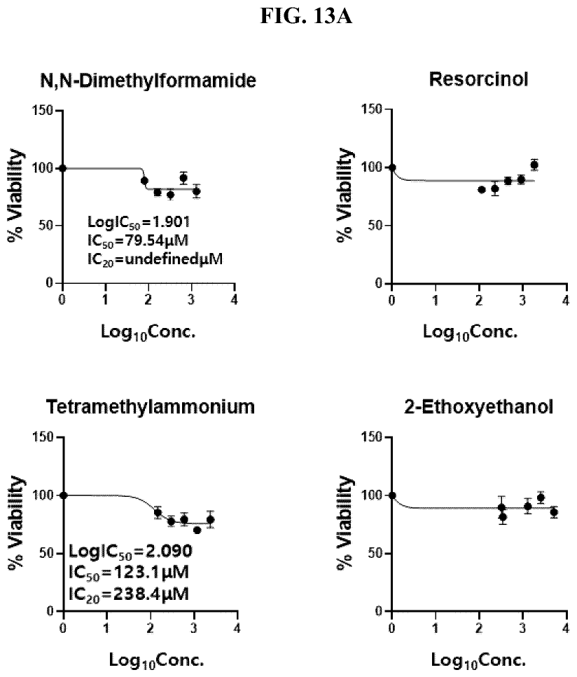

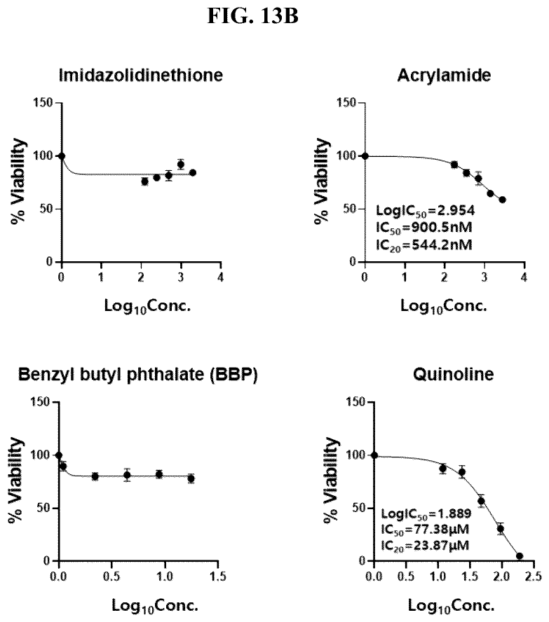

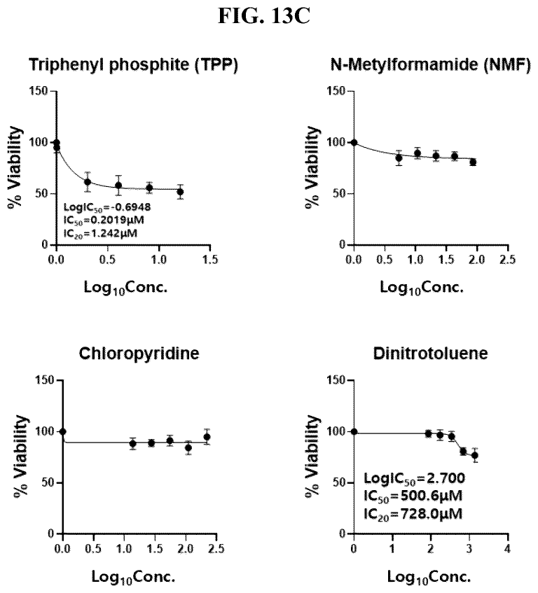

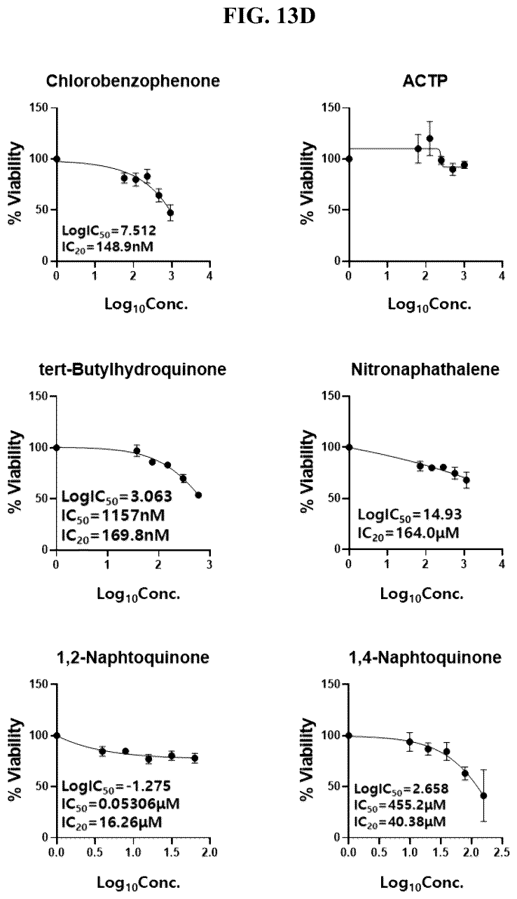

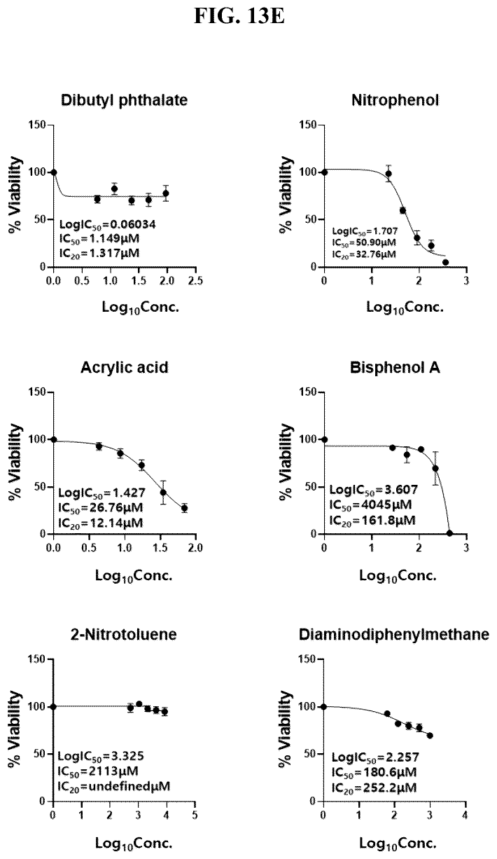

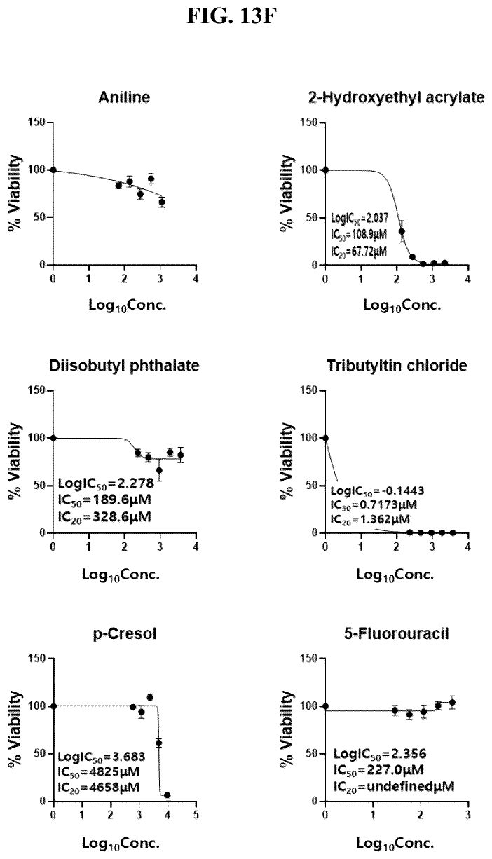

First, the CYP1A1 fluorescently labelled stem cell line-derived hepatic organoids according to the present disclosure were treated with the thirty substances for 24 hours to determine cell viability, and subsequent experiments were conducted by treating the substances at a maximum concentration that allowed the cells to survive. The change in cell viability according to the treatment of each substance is shown in FIGS. 13A-13F.

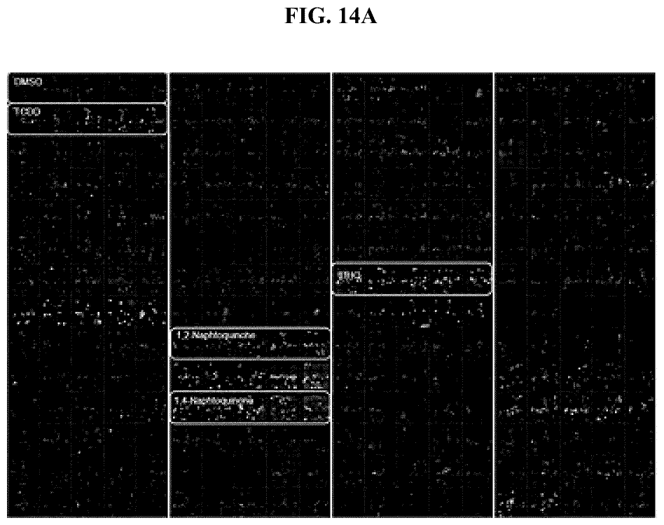

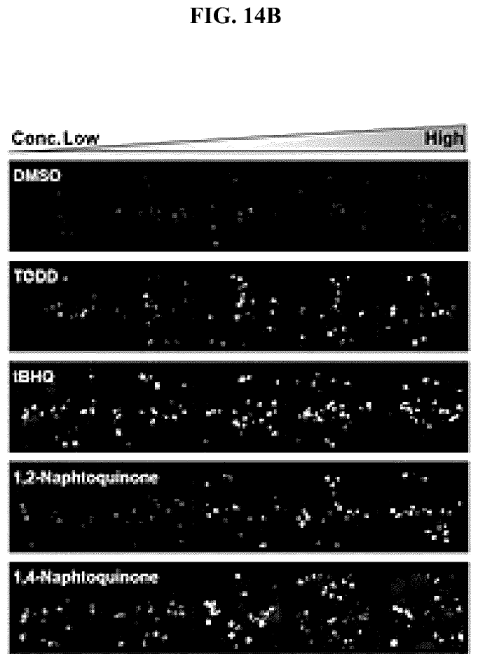

In addition, the hepatic organoids according to the present disclosure were treated with thirty substances to confirm the mCherry fluorescence intensity in real-time. As a result, as shown in FIGS. 14A and 14B, three hazardous chemicals with increased mCherry intensity could be selected.

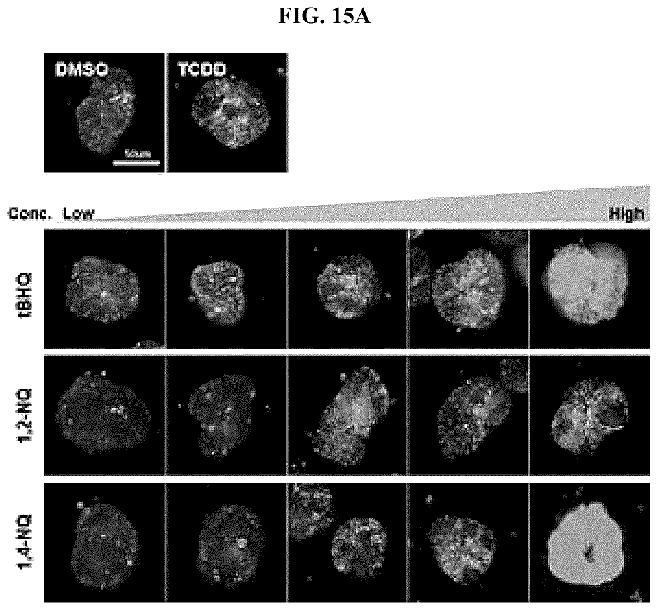

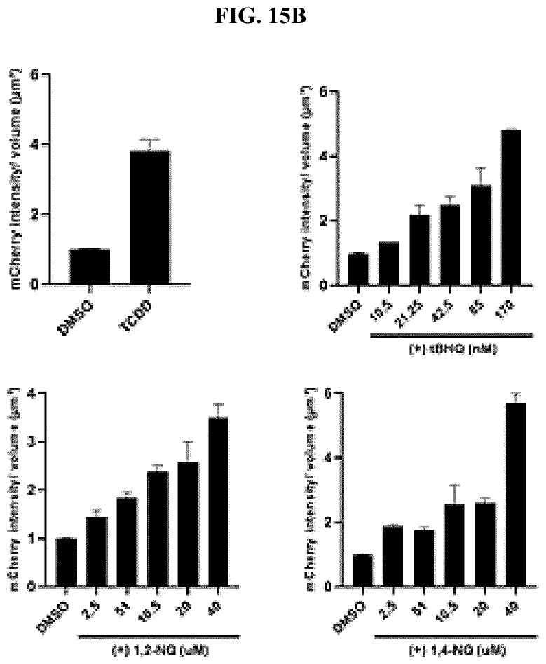

The CYP1A1 fluorescently labelled stem cell line-derived hepatic organoids were treated with the selected hazardous chemicals, tert-butylhydroquinone (tBHQ), 1,2-naphtoquinone (1,2-NQ), and 1,4-naphtoquinone (1,4-NQ) to observe the changes in mCherry fluorescence intensity in real-time. As a result, as shown in FIGS. 15A and 15B, it was confirmed that the fluorescence intensity was increased in a concentration-dependent manner of the substances (TCDD: positive control).

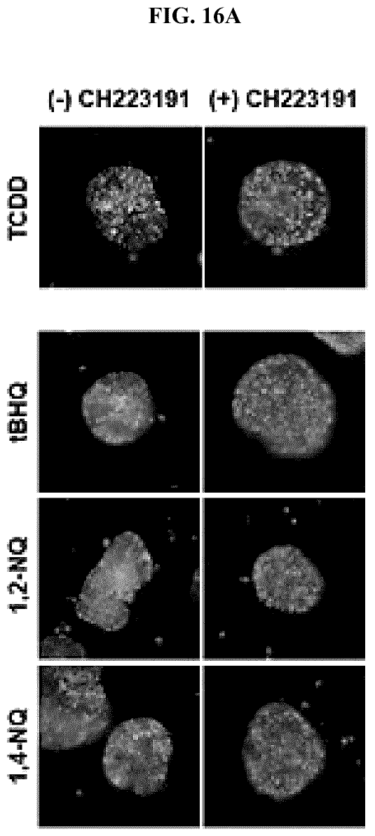

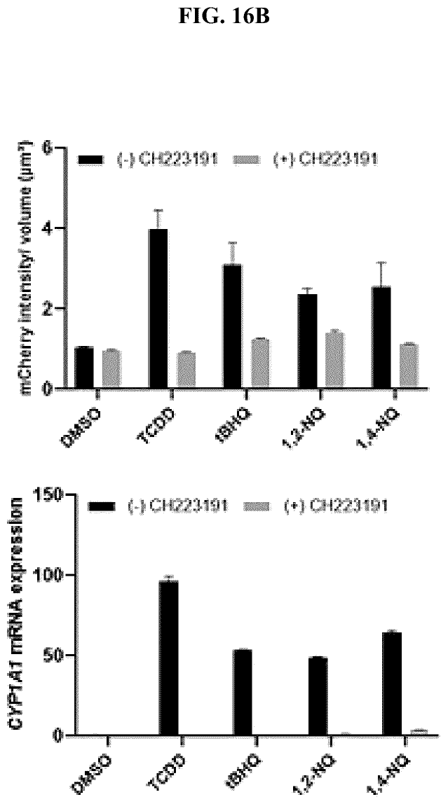

In addition, as shown in FIGS. 16A and 16B, it was confirmed that when three hazardous chemicals and the AHR antagonist CH223191 were treated together, the mCherry fluorescence intensity was decreased, which was consistent with the tendency to decrease the CYP1A1 mRNA expression level.

The method for screening an AHR modulators or a hazardous chemical using the CYP1A1 fluorescently labelled human pluripotent stem cell line-derived hepatic organoids according to the present disclosure enables the detection of substances that induce CYP1A1 regulation with higher sensitivity than when using the conventional human pluripotent stem cell line-derived hepatocytes by using hepatic organoids that can mimic the human body, and thus can be usefully utilized for early screening of new toxic or carcinogenic compounds while replacing animal testing.

From the above description, it will be understood by those skilled in the art to which the present disclosure belongs that the present disclosure may be conducted in other specific forms without altering its technical idea or essential features. In this regard, it should be understood that the embodiments described above are illustrative in all respects and are not intended to be limiting. The scope of the present disclosure should be interpreted that the meaning and scope of claims to be described below rather than the detailed description above, and all modifications or variations derived from the equivalent concept thereof are included in the range of the present disclosure.

What is claimed is:

1. A method for screening an aryl hydrocarbon receptor (AHR) modulator or a hazardous chemical, comprising:
(a) treating hepatic organoids derived from a human pluripotent stem cell line expressing cytochrome P450 fused to a fluorescent protein, with a test substance; and
(b) measuring a signal intensity of the fluorescent protein.

2. The method of claim 1, wherein the hepatic organoids express cytochrome P450 labelled with the fluorescent protein.

3. The method of claim 1, wherein the hepatic organoids derived from a human pluripotent stem cell are hepatic organoids matured by culture in a medium containing no extracellular matrix.

4. The method of claim 3, wherein the extracellular matrix is Matrigel.

5. The method of claim 3, wherein the culture in the medium containing no extracellular matrix is carried out using a bioreactor.

6. The method of claim 5, wherein a revolutions per minute (rpm) of the bioreactor is 50 to 90 rpm.

7. The method of claim 1, wherein the fluorescent protein is one or more selected from the group consisting of mCherry, green fluorescent protein (GFP), enhanced green fluorescent protein (eGFP), enhanced yellow fluorescent protein (EYFP), cyan fluorescent protein (CFP), mPlum, tdTomato, mStrawberry, J-Red, DsRed, mOrange, mKO, mScarlet, mKate, mBanana, mCitrine, Venus, YPet, Emerald, CyPet, Cerulean, and T-Sapphire.

8. The method of claim 7, wherein the fluorescent protein is mCherry.

9. The method of claim 1, wherein the human pluripotent stem cell line expressing cytochrome P450 fused to the fluorescent protein is prepared by transfection with a vector comprising a guide RNA targeting a sequence represented by SEQ ID Nos: 1 to 3.

10. The method of claim 1, wherein the cytochrome P450 is CYP1A1, CYP1B1, or CYP1A2.

11. The method of claim 1, further comprising (c) selecting a test substance with an altered signal intensity of the fluorescent protein compared to a control that was not administered the test substance.

12. The method of claim 11, the step (c) includes: determining that the test substance is an AHR agonist or a hazardous chemical if the signal intensity of the fluorescent protein indicates an increased level compared to a control that was not administered the test substance.

13. The method of claim 1, wherein the screening method is carried out while the cells are alive.

14. The method of claim 3, wherein the hepatic organoids are matured by culturing in the medium containing no extracellular matrix, hepatic endoderm organoids (HEOs) obtained by the following steps comprising:
(i) differentiating the human pluripotent stem cells (hPSCs) into definitive endoderm (DE) cells;
(ii) differentiating the definitive endoderm (DE) cells in the step (i) into hepatic endoderm (HE) cells; and
(iii) differentiating the hepatic endoderm (HE) cells in the step (ii) into hepatic endoderm organoids (HEOs).

15. The method of claim 14, further comprising (iv) subculturing differentiated hepatic endoderm organoids.

16. The method of claim 15, wherein a split ratio during the subculturing is 1:6 to 1:10.

17. The method of claim 14, wherein the hepatic endoderm organoids cultured in the medium containing no extracellular matrix are single cells.

18. A composition for screening an AHR modulator or a hazardous chemical, comprising hepatic organoids derived from a human pluripotent stem cell line expressing cytochrome P450 fused to a fluorescent protein.


SEQUENCE LISTING (3 sequences)

SEQ ID NO: 1 - DNA, 20 bp, synthetic construct
  source 1..20: mol_type = other DNA
gcattgatcctcctgtccat

SEQ ID NO: 2 - DNA, 20 bp, synthetic construct
  source 1..20: mol_type = other DNA
taggtagtggctcccttcaa

SEQ ID NO: 3 - DNA, 20 bp, synthetic construct
  source 1..20: mol_type = other DNA
ctggcactgacccctttgaa